(19) United States
(12) Patent Application Publication
(10) Pub. No.: US 2021/0205109 A1
(43) Pub. Date: Jul. 8, 2021

(54) FLEXIBLE ANKLE FOOT ORTHOSIS

(71) Applicant: Joseph W. Whiteside, Poland, OH (US)

(72) Inventor: Joseph W. Whiteside, Poland, OH (US)

(21) Appl. No.: 17/157,262

(22) Filed: Jan. 25, 2021

Related U.S. Application Data
(63) Continuation-in-part of application No. 16/123,410, filed on Sep. 6, 2018.

(51) Int. Cl. A61F 5/01; A61L 15/14; A61L 15/12

(52) U.S. Cl. CPC A61F 5/0111; A61L 15/12; A61L 15/14

(57) ABSTRACT

An ankle and foot orthosis of an integrated flexible yielding material to provide active or passive influence of foot and ankle position by direct adjustable elastomeric overlying strap. The orthosis extends over the lower limb having multiple strap attachment and tensioning points therealong with or without a non-yielding buildup extending heel and sole portion on which multiple strap fixation repositionable points are transversely positioned in spaced parallel, horizontal and diagonal orientation to one another and method of manufacturing the ankle and foot orthosis.

This continuation in part application claims the benefit of U.S. application Ser. No. 16/123,410, filed on Sep. 6, 2018.

### BACKGROUND OF THE INVENTION

### 1. Technical Field

This invention relates to ankle and foot orthosis that provide ankle and foot control stabilization needed to treat a variety of foot and ankle related conditions, and can indirectly treat knee, hip and truck position.

Orthosis are devices that provide support for a variety of supportive functional conditions such as plantar flexion and dorsiflexion, for example.

Orthotic devices of this type are custom made for specific patients to correspond to their anatomy or are selected from pre-formed basic orthotics which are then fitted to the individual user. As such, this method of fabrication is critical to the one-piece flexible ankle foot orthosis which requires multiple fabrication steps to be achieved.

### 2. Description of Prior Art

Prior art devices heretofore rely on piecemeal approach typically limited to their applied usefulness having rigid one-piece plastic moldings such as under the foot and over the orthosis with inherent structural imparted range of usefulness and a variety of disadvantages and limited applications.

Lower limb orthotic management using either a pre-fabricated or a custom Ankle Foot Orthosis (AFO) to provide stability, pain control and safe ambulation by assisting, resisting or stopping ankle and foot movement is very common. Typical candidates needing an AFO have lost all or some of their voluntary movement primarily about the ankle, resulting in the ankle and foot complex being improperly aligned. Having poor alignment associated with or without compromised voluntary movement can result in overloading and/or overuse of the soft tissue structures (muscle, tendon and cartilage, etc.) encompassing the ankle and foot complex. This overloading and/or overuse can result in excessive wear and tear to these structures and eventual changes to the bony structures, resulting in chronic pain and fixed deformities. The deformities and resultant malalignment many times results in further damage to the joints and structure (knee, hip and spine) proximal to the foot and ankle complex.

The challenge has always been and continues to be to optimally aligning the ankle and foot complex with an AFO having minimal bulk (not against the skin or very low-profile designs), simple to don and doff, while providing the necessary stability without restricting normal motion.

Examples of the prior art devices of this type have been developed with a variety of foot and ankle engagement structures, see for example U.S. Pat. Nos. 4,414,965, 6,117,098, 7,115,105, 8,100,845, and 8,734,371, as well as U.S. Patent Publications 2014/0288475 and 2008/0091131.

U.S. Pat. No. 4,414,965 discloses a brace for tibial fractures having a contoured portion with a plurality of straps extending in overlapping relation thereacross.

U.S. Pat. No. 6,117,098 is directed to an ankle brace having a fabric foot and ankle holder with multiple resilient bands in overlapping support thereon.

U.S. Pat. No. 7,115,105 claims a form fitting sock with a semi-rigid support member having a slot and lever motion stabilized strap as a lasso configuration thereabout.

U.S. Pat. No. 8,100,845 illustrates an ankle support with calcaneus control strap that wraps around the rear foot and mid foot to avoid excessive inversion and eversion of the foot.

U.S. Pat. No. 8,734,371 discloses a two-strap ankle brace with a non-rigid brace body and semi-rigid orthotic arch support.

In U.S. Patent Publication 2014/0288475, an ankle and foot orthosis has a tubular body around the foot and overlaps a second portion that extends partially along a dorsal aspect of the foot.

In U.S. Patent Publication 2008/0091131, a non-ambulatory thermo therapeutic boot for heat and cold therapy is disclosed showing a one-piece solid polymer gel pad with a strap extending thereabout.

### SUMMARY OF THE INVENTION

In accordance with the scope of the invention, an ankle and foot orthosis is disclosed that addresses multiple shortcomings of traditionally available orthosis devices (which are typically not flexible enough to externally influence the ankle and foot position with an elastic strap).

A flexible one-piece thermoplastic body member, total contact ankle and foot orthosis of the invention, includes a positional elastic strap repositionable along the planar support surface to multiple cross strap attachment points having the capability of resisting the tension extended thereby without deformity while distributing the force over a large surface area (allowing for increased or decreased dorsiflexion assist or plantar flexion resist by repositioning or increasing tension between the strap securing points on the medial and lateral side to its crossing points).

### DESCRIPTION OF THE DRAWINGS

FIG. 1 is a medial side elevational view of the ankle foot orthosis of the invention with support strap engaged.

FIG. 2 is a lateral side elevational view thereof.

FIG. 3 is a medial side elevational view of the ankle and foot orthosis without support strap.

FIG. 4 is a lateral side elevational view thereof.

FIG. 5 is an enlarged bottom plan view with the strap portion shown in broken lines.

FIG. 6 is a front elevational view of the ankle and foot orthosis.

FIG. 7 is a rear elevational view of the ankle and foot orthosis.

FIG. 8 is a top plan view of the ankle and foot orthosis of the invention without straps.

FIG. 9 is a side elevational view of an alternate strap engagement retainment assembly.

FIG. 10 is a partial perspective view of an alternate strap and buckle retainment assembly.

FIG. 11 is a block flow diagram of the fabrication steps for the ankle and foot orthosis of the invention.

FIG. 12 is a side perspective view of the orthosis formation on a vacuum mold.

FIG. 13 is a perspective side view of the orthosis formation during mold released trimming.

FIG. 14 is an enlarged partial perspective view of the molded orthosis with edge finishing tool engaged thereon.

FIG. 15 is a side perspective view of the ankle and foot orthosis illustrating strap placement strips and initial elastic strap placement thereabout.

### DETAILED DESCRIPTION OF THE INVENTION

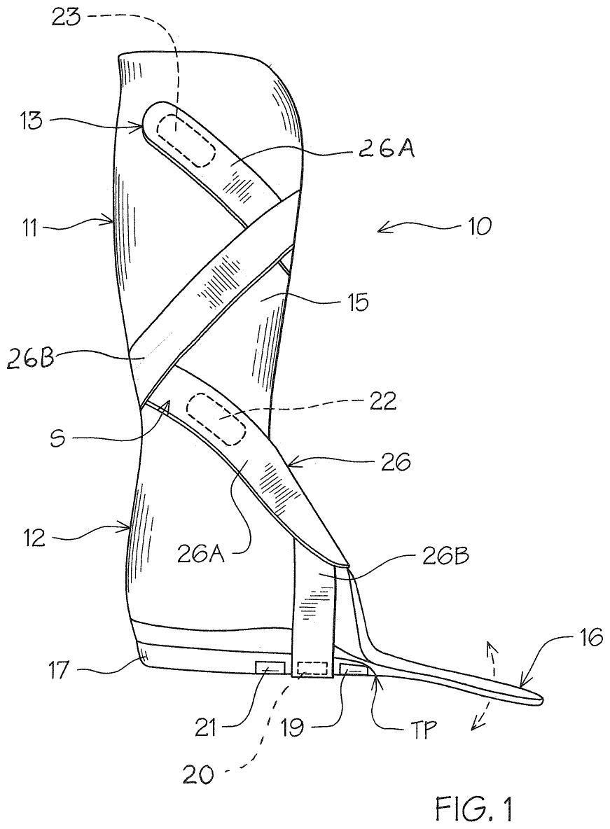

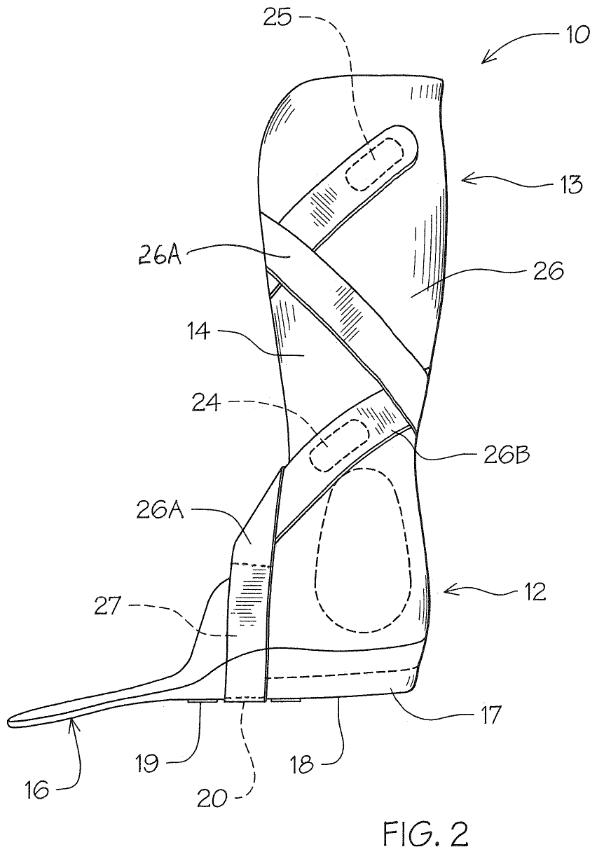

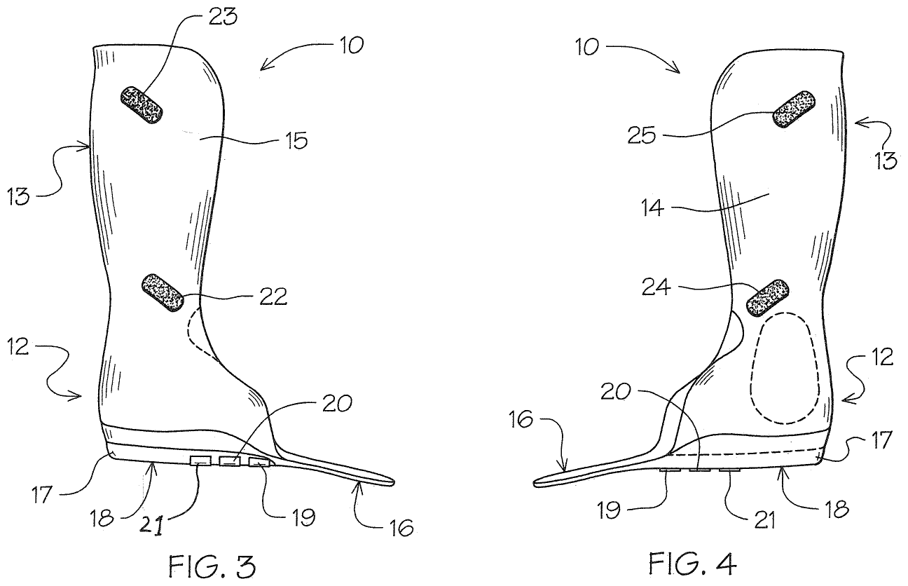

Referring to FIGS. 1 and 2 of the drawings, the ankle and foot orthosis 10 of the invention can be seen having a one-piece flexible thermoset or thermoplastic split body member 11 with a heel and foot engagement portion 12 and an ankle upper leg engagement portion 13. The thermoplastic body member 11 is custom made, custom fit or prefabricated as best seen in FIGS. 3, 4 and 6 of the drawings formed with flexible medial right and lateral left sides 14 and 15 with an aligned general effacing open perimeter edges 14A and 15A as best seen in FIG. 6 of the drawings.

It will be evident that an alternate embodiment may include, as noted, a prefabricated or customizable ankle and foot orthosis having the same flexible one-piece unique yielding thermoplastic body member in pre-formed incremental size variance that can be correspondingly modified, as required, for use. An example of such thermoplastic material with preferred resilient and limited yielding properties can be found in ethylene vinyl acetate copolymer based synthetic resin materials commercially available under trade names from a group including Proflex®, Duraflex® and OP-TEX Flex®, all of which are well known and available in the thermoplastic art.

The respective right or medial and left or lateral sides 14 and 15 extend integrally from one another along the heel and foot engagement portion 12 and define a flexible extending front foot base support 16 again best seen in FIG. 6 of the drawings. The integrated heel and foot engagement portion 12 has a rigid buildup heel wedge 17 that extends along a sole portion 18 up to the flexible front foot, can be with or without a “flexible front base”, support 16 defined starting at transition from the heel wedge 17 (i.e from the transition point generally at 19) to the very end of the foot base support 16.

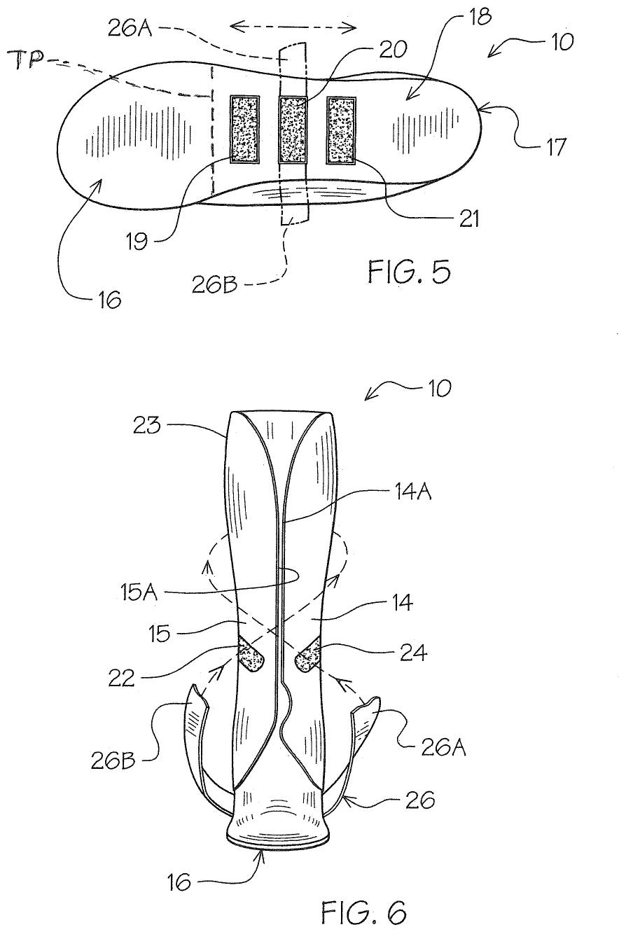

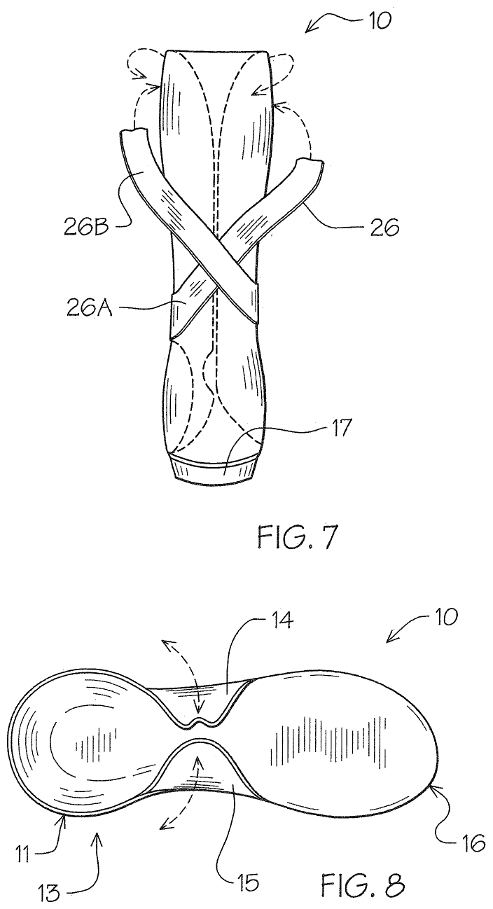

Multiple strap engagement strips 19, 20 and 21 of fabric strap engagement or other type of securing system retainment material, described hereinafter, are in spaced parallel, horizontal and/or diagonal relation to one another extending transversely along the sole portion 18 emanating from about the foot support transition point TP (seen in FIG. 1) as best illustrated in FIG. 5 of the drawings.

Each of the body member 11 flexible extending sides 14 and 15 have a pair of engagement retainment strips 22, 23 and 24, 25 respectively in spaced longitudinal relation to one another that as hereinafter described will adjustably retain a dynamic correction strap 26 when positioned thereover.

Each of the strap engagement retainment strips 22-25 and strap engagement strips 19-21 are, in this example, of fabric hook and loop materials known commercially as Velcro® and the strips 19-21 and 22-25 are secured to the thermoset or thermoplastic material at the sole portion 18 and respective sides 14 and 15 by adhesive or other method of securing.

The engagement restraint strips 22-25 can be positioned adjacent, more forward (anterior) or back (posterior) to the respective right and left side perimeter edges 14A and 15A clearly illustrated in FIGS. 3 and 4 of the drawings for proportional dynamic strap engagement as will be disclosed in greater detail hereinafter.

The dynamic correction strap 26 is preferably of a strip of elastic polyamide, elastomer-based hook and loop material having a smooth support surface S and a continuous strap engagement “loop” LS in compliance with hook and loop attachment criteria well known within the art as Velstretch® strapping and as hereinbefore described.

The dynamic correction strap 26 is initially selectively engaged across one of the strap engagement strips 19, 20 or 21 depending on the positional resistance or restraint needed with equal strap lengths 26A and 26B extending respectively from their respective sides. The bands lengths 26A and 26B are preferably equal and extend up and crisscross over the lower foot portion before wrapping around the respective oppositely disposed sides 14 and 15 in their orientation then overlyingly engaging and retained on the first of the corresponding side engagement retainment strips 22 and 24, best seen in FIGS. 1, 2 and 6 of the drawings.

The respective strap lengths 26A and 26B then wrap around the body member 11 and crisscross again on the ascending upper leg back portion to the opposite sides 14 and 15 engaged and selectively retained on the remaining engagement retainments 25 and 23 respectively.

This unique combination of the flexible resilient body member 11 and the elastic strap 26 provides a multitude of dynamic adjustment supportive features as will be outlined hereinafter.

The instant invention has the ability to increase or decrease dorsiflexion assist or plantar flexion resist by repositioning the strap 26 on the plantar aspect of the foot. Increasing dorsiflexion assist and resisting plantar flexion is accomplished by releasing the strap 26 and moving the strap distally on the foot sole portion 18. With reference to FIG. 5 of the drawings, changing the strap 26 position from engagement strip 21 to 20, in this example, may achieve optimum assist or resist for the control, stabilization and/or treatment needed.

Dorsiflexion or plantar flexion resist can further be increased by stretching and repositioning (i.e., increasing the tension of the straps 26A and 26B from the corresponding strap engagement strips 19, 20 and 21), in this example, to the respective opposite side engagement restraints 22 and 24 indicated by directional orientation arrows in FIG. 6 of the drawings.

It will therefore be seen that additional adjustment can consist of adjusting the tension either proportionally or disproportionally to match the ideal tension necessary to stabilize or assist the amount of dorsiflexion, plantar flexion, supination and pronation to provide proper alignment, assist, resist and/or stability for individual patient's need.

The adjustability of the tension by the repositioning and retaining of the effective strap lengths 26A and 26B about the flexible thermoplastic one-piece split body member 11 is achieved in part to the effective material thickness which, in this example, is defined by ranges of 0.125, 0.1562, 0.1875, and 0.250 inches or proportionally thicker depending on application required.

Further stabilization and assistance and/or resistance to the amount of dorsiflexion, plantar flexion, supination and pronation can be achieved by adding to the thermoset or thermoplastic material, in this example, a section of non-yielding synthetic fiber material 27 (e.g. Dacron®) between two points of the elastic straps 26A or 26B illustrated specifically in FIG. 2 of the drawings shown in broken and dotted lines.

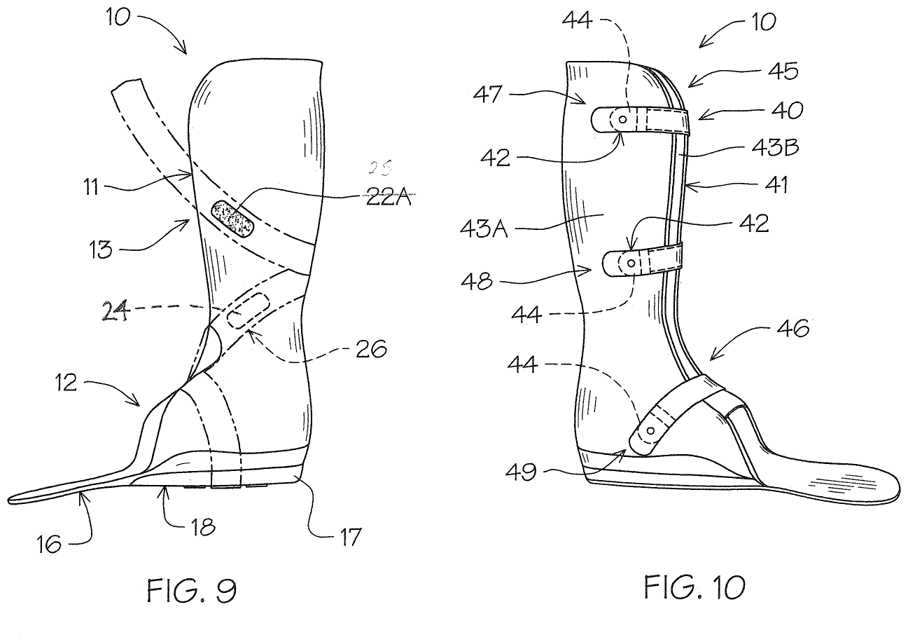

While the above disclosed strap configurations can induce internal and/or external rotation of the shank, additional tension, resistance, assistance can be achieved by adding an additional or supplemental strap engagement resist 23, 25 on the medial and/or lateral mid-calf area of the upper leg engagement portion 13 with the strap illustrated in broken lines thereabout, as seen in FIG. 9 of the drawings.

Referring now to FIG. 10 of the drawings, an alternate strap attachment assembly 40 can be seen generally in which a split flexible body member 41 with an integrated leg, ankle and foot portion has a series of longitudinally spaced strap attachment fixation points 42 which are positioned on the corresponding flexible upstanding sidewalls 43A and 43B thereof with adjusting strap engagement buckle assemblies 44 on opposite sidewall 43B so as to provide multiple horizontal transverse partial extension between respective upper leg 45 and integral body member and ankle and foot portions 46 in a point to point manner. The so configured retainment engagement assemblies 47, 48 and 49 are therefore in translateral aligned corresponding orientation on the opposing body sides thereby adjustably securing the respective sides of the alternate split flexible body member 41 about the user, not shown.

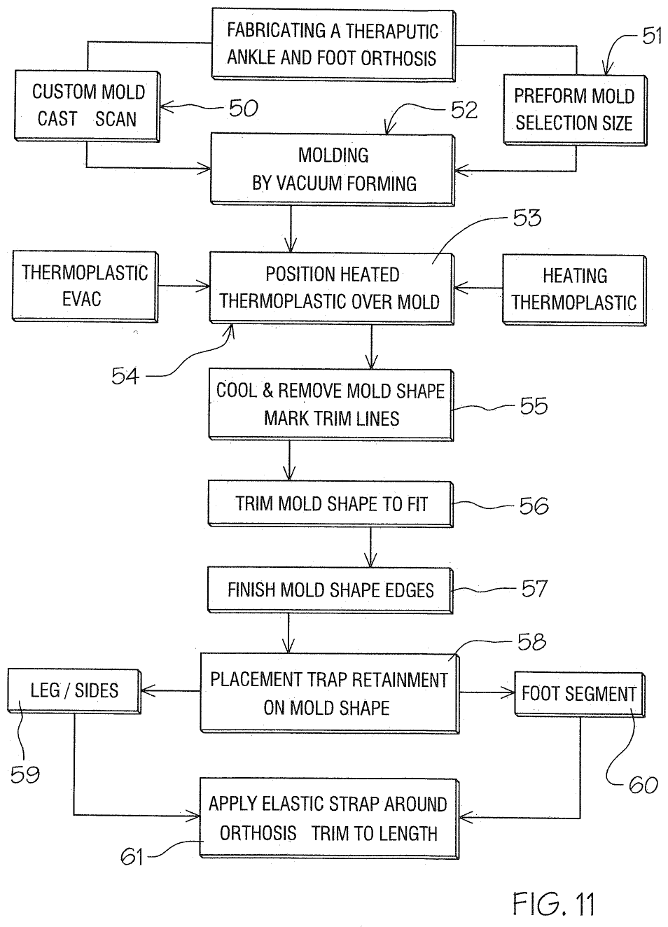

Referring now to FIGS. 11-15 of the drawings, multiple fabrication and finishing steps are shown for both a custom and an “off the shelf” and size preferred ankle and foot orthosis of the invention.

Referring now specifically to FIG. 11 of the drawings, a block flow diagram can be seen illustrating the fabrication steps for a custom mold defined orthosis and a select size pre-formed mold base orthosis.

A custom mold at step 50 is achieved by any one of the commercially known and established techniques including, but not limited to a direct patient cast or a 3D scan to form a positive mold in this example.

A pre-formed orthosis mold at step 51 provides multiple universal mold configurations in different sizes from x-small progressively to 2X large in either pediatric or adult size from which to select from.

It is noted that utilization of the pre-formed select side molds 31 will provide for reduced cost and therefore greater availability than the “one off custom molds”.

In use of either the custom mold or the selected side pre-form mold will both require that the molding step 52 uses heated thermoplastic sheet material PSM to be positioned over the so determined positive mold at step 53. Anyone of the known thermoplastic materials may be used such as ethylene vinyl, acetate copolymer (EVAC) available under the trade names Proflex®, Duraflex®, and OP-TEX Flex® as hereinbefore described. EVAC has inherent flexible properties better maintained even after molding.

The molding process used in this example is by vacuum forming at step 54 that uses applied vacuum to induce negative air pressure to a so positioned and heated thermoplastic sheet on selected mold, in this example.

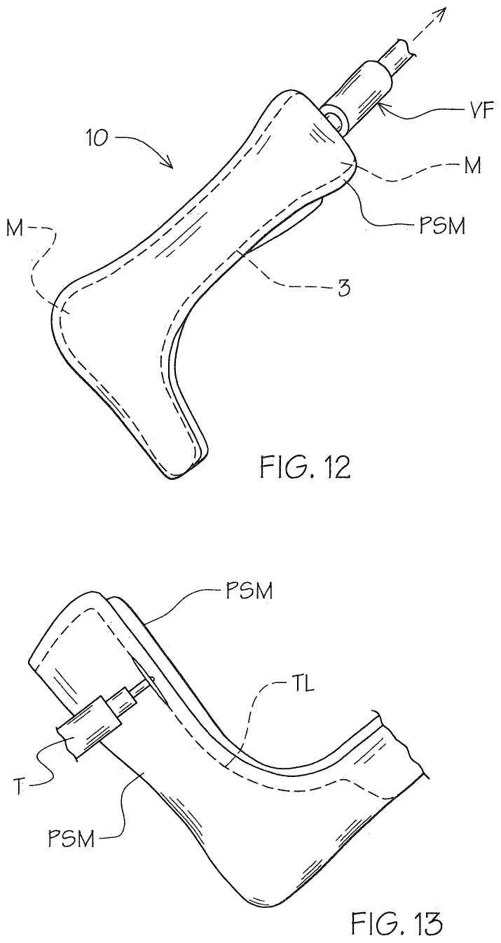

Such vacuum forming VF of the thermoplastic sheet PSM, see also FIG. 12 of the drawings, over a positive mold M, shown in broken lines, is well established in the art with a heating range of material of between 250- and 700-degrees Fahrenheit, in this example.

The thickness of the thermoplastic range typically used is 0.125 (⅛^{th}), 0.15625 ( 5/32^{nd}), 0.1875 ( 3/16^{th}), and 0.25 (¼^{th}) of an inch which will afford ease of vacuum forming of contoured about the selected mold M. Referring also now to FIG. 13 of the drawings, the cooled basic mold orthosis is fixed and released at step 55 which includes marking trim lines TL thereon.

The trim lined marked orthosis shape is then trimmed at step 56 and seen graphically in FIG. 13 of the drawings by, in this example, a rotary trimmer T following the trim lines TL thereby taking on the contour and proper predetermined dimensions of the flexible ankle and foot orthosis 10 of the invention.

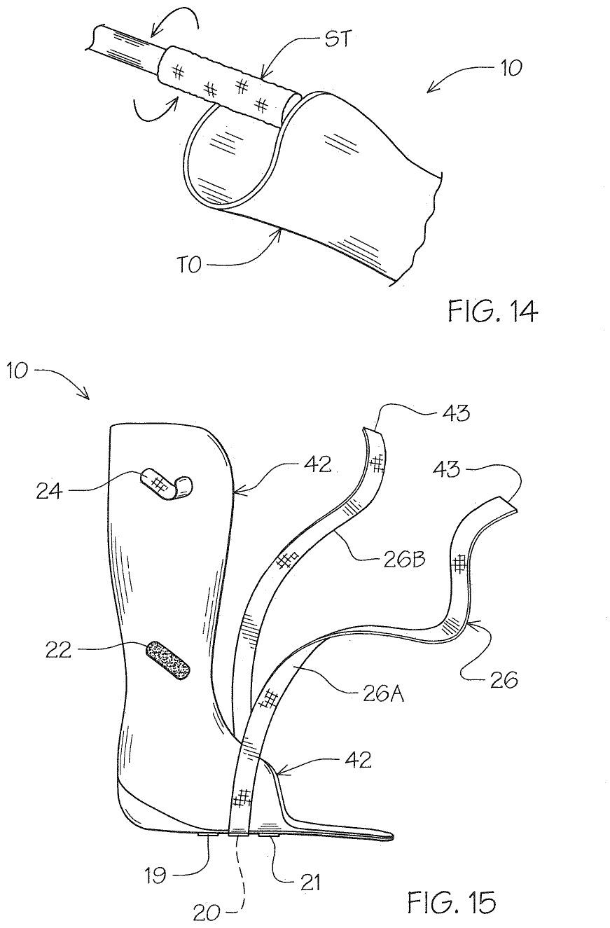

The trimmed orthosis shape TO is then edged finished at step 57 as seen in FIG. 14 of the drawings using a rotary sanding tool ST.

Completion of the fabrication steps requires placement of multiple hook and loop strap retaining strips 19-25 as identified in step 58 and illustrated in FIG. 15 of the drawings on the respective upper leg engagement portion 13 and heel and foot engagement portion 12. The single dynamic correction strap 26 is placed around the orthosis shape at step 61 and over the trimmed edges 14A, 15A at step 61 illustrated both in FIGS. 11 and 15 of the drawings and trimmed to length at TL.

It will thus be seen that a new and novel flexible ankle and foot orthosis 10 has been illustrated and described that provides a number of dynamic configurations depending on materials and securing points which can be selected to assist or resist dorsiflexion and/or plantar flexion through adjustable desired assist, resist stabilization/stop or limit supination and/or pronation and stabile/stop or limit internal or external rotation of the shank (i.e. body segment between the knee and the ankle). The device has been designed to provide stability to the lower limb and all planes of motion by encompassing the foot, ankle and calf segments in a unique and novel strap orientation and structural configuration, resulting in improved balance and sensory input throughout the gait cycle.

It will thus be seen that various changes and modifications may be made therein without departing from the spirit of the invention. Therefore, I claim:

1. A therapeutic ankle and foot orthosis comprising:
a flexible and resilient, one-piece thermoplastic ankle and foot engagement body member, the ankle and foot engagement body member being custom contoured having an elongated split opening being closable for a full contact fit for a patient's ankle and foot;
a single elastic strap being selectively repositionable transversely along a planar underside support surface of a foot portion of said ankle and foot engagement body member, the elastic strap having a pair of free ends extend from the planar underside support surface of the foot engagement portion in opposing spaced opposition to one another in multiple crisscross engagements about said ankle and foot engagement body member; and
pairs of strap engagement and retainment strips secured in longitudinally spaced parallel angular orientation to one another on respective outer opposing surfaces of said ankle and foot engagement body member for positionable engagement and retainment of said crisscrossed strap there along and about.

2. A method of manufacturing an ankle and foot orthosis comprising the steps of,
selecting a mold suitable for a patient from a group including, a custom mold, and pre-formed molds of different sizes,
positioning a deformable sheet of thermoplastic over the selected mold,
applying heat to the thermoplastic sheet and molding to conform around the selected mold defining an orthotic formed shape,
removing said orthotic formed shape from said selected mold,
trimming said orthotic formed shape to fit the patient,
placing a plurality of material retainment strips on the trimmed orthotic formed shape for selectively retaining an elastic strap there about,
positioning the elastic strap around the trim orthotic formed shape.

3. The method of manufacturing the ankle and foot orthosis set forth in claim 2 wherein said custom mold is formed from a direct impression along a portion of a patient's leg and complete ankle and foot.

4. The method of manufacturing the ankle and foot orthosis set forth in claim 2 wherein said thermoplastic sheet is flexible before and after molding.

5. The method of manufacturing the ankle and foot orthosis set forth in claim 2 wherein molding said thermoplastic to conform around the selected mold comprises,
heating said thermoplastic sheet and applying a vacuum to said selected mold.

6. The method of manufacturing the ankle and foot orthosis set forth in claim 2 wherein said trimming the orthotic formed shape comprises,
cutting along a trim line on the orthotic shape and finishing cut edges of the orthotic shape.

7. The method of manufacturing the ankle and foot orthosis set forth in claim 2 wherein placing a plurality of material retaining strips on said trimmed orthotic formed shape comprises,
securing said retaining strips individually on a sole portion and respective medial right and lateral left outer sides of the orthotic formed shape in spaced relation to one another.

8. The method of manufacturing the ankle and foot orthosis set forth in claim 2, wherein said selectively retaining said elastic strap and retainment strips comprises, hook and loop material.

9. The method of manufacturing the ankle and foot orthosis set forth in claim 2 wherein positioning the elastic strap around the trimmed orthotic formed shape comprises,
wrapping the elastic strap in double crisscross overlapping pattern there about.

10. The method of manufacturing the ankle and foot orthosis set forth in claim 2 wherein said deformable sheet of thermoplastic is of a synthetic resin material having a thickness in the range of ⅛^{th}, 5/32^{nd}, 3/16^{th} and ¼^{th} of an inch.
